(19) United States
(12) Patent Application Publication
(10) Pub. No.: US 2009/0224126 A1
(43) Pub. Date: Sep. 10, 2009

(54) Retaining device and microscopic arrangement

(76) Inventor: Daniel Kolster, Oberkochen (DE)

Correspondence Address: KRIEGSMAN & KRIEGSMAN, 30 TURNPIKE ROAD, SUITE 9, SOUTHBOROUGH, MA, 01772, US

(21) Appl. No.: 12/378,619

(22) Filed: Feb. 18, 2009

(30) Foreign Application Priority Data
Feb. 18, 2008 (DE) 10 2008 009 678.4

(51) Int. Cl. G02B 21/26; A61B 19/00; F16M 11/12; G02B 7/00

(52) U.S. Cl. 248/324

(57) ABSTRACT

Among other things, a retaining device (10) for tilting and swiveling an apparatus, in particular a microscope, which is fastened to retaining device (10), is described, which has a fastening means (11) for fastening the apparatus, a tilting axis (12) for tilting the fastening means (11), and a swiveling axis (13) for swiveling the fastening means (11). In order to reduce the necessary structural space and to be able to guide cables through the axes, it is provided that the tilting axis (12) and the swiveling axis (13) lie in one plane and are aligned with one another in such a way that they intersect. Tilting and swiveling can be realized with the help of a specially configured gear (14). Retaining device (10) advantageously has two drives (15: 18) that can be actuated independently of one another. The drive direction of drives (15; 18) alone decides whether the movement is a tilting movement, a swiveling movement or a combined tilting/swiveling movement. In addition, a microscope arrangement is also described.

The present invention first relates to a retaining device for tilting and swiveling an apparatus, in particular a microscope, which is attached to the retaining device, according to the preamble of patent claim 1. Further, the invention also relates to a microscopic arrangement.

Retaining devices, to which apparatuses are attached in a tiltable and pivotable manner, are already known in the most diverse configurations. For example, such apparatuses may be microscopes, e.g., operating microscopes and the like. In the further course [of the description], the invention of the offerer [Applicant] will be described on the basis of microscopes, and here particularly based on operating microscopes, but the invention is not limited to this as a specific application.

For example, operating microscopes must be freely positionable over the operating field. It was previously common to always conduct the tilting movements and swiveling movements performed in this way in two separate axes. Such a solution is described, for example, in DE 102 60 888 A1. A microscope mount described therein comprises a fastening means for fastening a microscope, an axis of rotation that makes possible a rotation of the microscope, and a swiveling axis, which makes possible a swiveling of the microscope. In addition, a tilting axis is also provided, so that the microscope can be tilted. The individual axes are thus disposed spatially separated from one another. A large structural space is necessary for such a configuration, which concerns a swiveling and tilting of the microscope.

Proceeding from the named prior art, the object of the present invention is to further develop a retaining device of the type named initially, so that the structural space necessary can be reduced. In addition, a correspondingly improved microscope arrangement shall be provided.

This object will be solved according to the invention by the retaining device with the features according to the independent patent claim 1 as well as the microscope arrangement with the features according to the independent patent claim 16. Other features and details of the invention can be taken from the subclaims, the description and the drawings. Features and details that are described in connection with the retaining device according to the invention, of course, are valid also in connection with the microscope arrangement according to the invention, and vice versa.

According to the first aspect of the invention, a retaining device for tilting and swiveling an apparatus, in particular, a microscope, which is attached to the retaining device, is provided, having a fastening means for fastening the device, a tilting axis for tilting the fastening means, and a swiveling axis for swiveling the fastening means. The retaining device is characterized according to the invention in that the tilting axis and the swiveling axis lie in one plane and are aligned with one another in such a way that they intersect.

A compact tilting and swiveling mechanism will be realized by the retaining device according to the invention, which can be used particularly in connection with microscopes, for example, operating microscopes. In contrast to this, the previously known retaining devices all require a larger structural space, since the axes are spatially separated from one another. Elements necessary for operation, such as, for example, cables and the like, had to always be laid through the two axes or had to be laid past these axes in the known solutions. The structural space necessary can now be considerably reduced by the configuration of the retaining device according to the invention. In addition, the cables can be simply and comfortably guided through the axes.

The retaining device according to the invention first provides a fastening means, to which the apparatus to be tilted and swiveled is fastened. In connection with a microscope, the fastening means can be a microscope mount, for example. A tilting axis around which the fastening means can be tilted as well as a swiveling axis around which the fastening means can be swiveled are also provided.

In this way, the necessary structural space can be reduced, since it is now provided that both axes lie in a single plane. Further, it is provided that the two axes intersect. Advantageously, it can be provided that the two axes are aligned perpendicular to one another in one plane. Additionally, with this configuration, the drive device for the tilting process and the swiveling process can also be very greatly simplified, which translates to a further advantage of the retaining device according to the invention. Non-exclusive examples and the particulars of how these can be realized will be explained in more detail in the further course of the description.

Advantageously, in order to conduct a tilting movement of the fastening means around the tilting axis and in order to conduct a swiveling movement of the fastening means around the swiveling axis, a single gear can be provided. The invention is not limited to specific embodiments for the gear, however. Generally, the gear may involve any mechanical device for transferring and changing rotating movements, directions of rotation and torque. For example, the gear that is used may involve a type of driven universal gear in the form of an inverted differential gear or similar gear.

Advantageously, the retaining device can have two actuatable drives that are independent of one another, by means of which a tilting of the fastening means around the tilting axis and a swiveling of the fastening means around the swiveling axis can be carried out. Therefore, two drives carry out the tilting movement, the swiveling movement or a combined movement of the apparatus fastened to the fastening means.

It is advantageously provided that the two drives are designed so that they can be actuated in a synchronous or asynchronous manner.

In this way, a situation may occur in which only one drive is operated in a specific drive direction, while the other drive is not actuated. It is likewise possible that both drives will be actuated simultaneously. For example, a situation may occur, in which both drives will be synchronously actuated, i.e., with the same drive direction. Likewise, a situation may occur in which both drives will be asynchronously actuated, i.e., in opposite drive directions.

For example, it may be provided that the two drives can be operated with different directions of rotation. Each of the drives can have different directions of rotation, considered in and of itself, i.e., can be operated with different directions of rotation. Likewise, it is conceivable that the two drives have the same and/or different directions of rotation, in comparison to one another. If the two drives rotate synchronously, i.e., in the same direction, then, for example, a tilting movement of the apparatus, e.g. of the microscope, can be carried out. If the two drives do not rotate synchronously, for example, if one drive rotates counter to the other drive and preferably to the same extent, then the apparatus carries out a swiveling movement. If only one of the drives rotates, then the apparatus carries out a combined tilting and swiveling movement.

The drive direction of the drive by itself, for example, its direction of rotation, then decides whether the movement is a tilting movement, a swiveling movement or a combined movement in the form of a tilting/swiveling movement.

Preferably, the retaining device has a bearing block, through which are extended the tilting axis and the swiveling axis. This bearing block can be disposed, for example, inside the gear. It is then possible, for example, to guide cables and the like through the center. The bearing block serves to support the necessary bearings, which may involve, for example, axial bearings, radial bearings, combined axial/radial bearings, or the like.

The present invention is not limited to specific embodiments of how the tilting axis and the swiveling axis have been designed. It may be advantageously provided that the tilting axis and the swiveling axis are designed as structural pieces joined with the bearing block. In this way, the structural pieces can be joined with the bearing block so that they can be detached or cannot be detached, as needed. In a preferred embodiment, it may be provided, for example, that the pieces are each designed as screw-threaded pieces that can be screwed into the bearing block and can be screwed together with the block in a fixed manner.

In order to be able to transfer rotational movement to the gear, each drive has a worm drive in a preferred configuration. The worm drive, in turn, can be driven via a suitable motor.

Advantageously, the gear can have two worm wheels, which are disposed along the tilting axis. Each drive then propels one worm wheel. For example, it may be provided that a first drive comprising a first motor and a first worm drive, propels a first worm wheel, while a second drive, comprising a second motor and a second worm drive, propels the second worm wheel. Therefore, each of the worm wheels can be driven individually and independently from every other worm wheel. Advantageously, the worm wheels are disposed along the tilting axis, so that the other elements of the gear are found between the two worm wheels. This applies particularly to the bearing block and the fastening means for the apparatus, e.g., the microscope.

In another configuration, each of the worm wheels can be joined with a drive bevel wheel. For example, it may be provided that a worm wheel and a drive bevel wheel can be designed as a single component. If the worm wheel and the drive bevel wheel are rigidly joined with one another, the latter may also be present as two individual components.

It may be particularly provided that the drive bevel wheels are mounted in the bearing block.

It may be preferably provided that the gear has a driven bevel wheel, which is rigidly joined with the fastening means. Here, it may be particularly provided that the driven bevel wheel is disposed along the swiveling axis. The driven bevel wheel is particularly joined in place with the fastening means, for example, a microscope mount. Advantageously, the driven bevel wheel is designed and disposed in such a way that it can mesh with the drive bevel wheels that have been described further above.

In another configuration, the gear may have a counter bevel wheel, which is mounted on the bearing block. This counter bevel wheel may also be disposed advantageously along the swiveling axis. This is particularly advantageous if the driven bevel wheel, which has been described further above, and the counter bevel wheel are asynchronous. The counter bevel wheel is only optional and also may be omitted or may be replaced by a mounted shaft.

Preferably, the retaining device may have an apparatus for determining position. The position of an apparatus, for example, a microscope, in particular an operating microscope, which is fastened to the retaining device, can be determined by means of this apparatus for determining position.

There are various possibilities to be able to determine the position of the apparatus fastened to the retaining device, so that the invention is not limited to specific configurations of the apparatus for determining position. Several advantageous, but non-exclusive examples will be described below for this purpose.

For example, the apparatus for determining position may be designed as an encoder device, advantageously as a magnetic encoder device. It is possible, for example, by means of such an encoder device, to determine the position of individual wheels relative to one another, for example, the worm wheels, as described above.

Thus, one possibility is detecting the position of the worm wheels relative to one another, for example, by means of a magnetic encoder device. Here, a magnet, which is found above each sensor element (for example, a sensor IC), is joined in a fixed manner with the respective worm wheel. By interpolating the two encoder values, the position of the apparatus fastened to the retaining device can be determined. Further possibilities for apparatuses for determining position are, e.g., potentiometers, each of which is preferably integrated in a drive unit/gear unit. If a reference travel is allowed, an incremental transmitter (not absolute) can be used on the drives for determining position.

The position of the apparatus e.g., a microscope, in particular, an operating microscope, which is fastened to the retaining device, however, can also be determined in another way, for example, directly, with two angle sensors disposed at a right angle, such as are used, for example, in electronic level detectors. In such a case, of course, the position determination is made directly in the apparatus, so that in this case, the apparatus for determining position need not be a component of the retaining device, but also may be a component of the corresponding apparatus, e.g., the microscope, in particular, the operating microscope.

Advantageously, the retaining device may have an apparatus for fastening to a stand. The stand serves for the uptake, in particular, for the moveable uptake, of the corresponding apparatus, e.g., the microscope, in particular, the operating microscope. Each time depending on the configuration, the retaining device may also involve the stand itself, of course, or a component of such a stand.

By means of a suitable selection and combination of the gear as well as the individual gear members, the gear preferably can be designed as a self-locking or non-self-locking system. Self-locking gears are basically characterized in that they can keep a driven axis in a driven position and that the driven axis can only be rotated by a rotation of the drive shaft. In a non-self-locking system, the apparatus fastened to the fastening means, e.g., a microscope, could be equilibrated, for example, with a spring or spring-type device, or according to the principle of an electronic spring, and/or by means of electronic brakes, and be freely positioned by hand. Worm gears may be used advantageously for a self-locking system. It is also conceivable, however, that instead of worm wheels, toothed wheels may also be used, as long as the torque that occurs can be compensated, for example, by motor brakes or the like. This configuration is then advantageous, for example, if one would like to control or actuate the brakes by pressing a button in order to manually position the apparatus, for example, the microscope.

The retaining device according to the invention further offers the possibility of guiding cables and the like through the two axes at the same time. In this case, the cables will be bent advantageously along the neutral axis without lengthening or shortening.

An essential advantage of the retaining device according to the invention with the special drive and the special gear consists of the fact that twice the components will be used. In this way, not only can money be saved, but the individual components may also be designed smaller due to the [increased] strength. The torque, which acts on the tilting axis, is divided, for example, onto the two worm wheels. Thus, it is not just one worm wheel that bears the torque, but two. This has enormous effects on structural size. If a counter bevel wheel is used, then continually more teeth are engaged in order to take up the torque, instead of just one, if the tilting process is considered. This permits a smaller tooth module or reciprocal of the diametral pitch. The direction of rotation of the two drives alone determines the tilting or swiveling movement.

In an advantageous embodiment, the retaining device can have two driven drive bevel wheels, as well as a driven bevel wheel, which engages in both driven drive bevel wheels. The bevel wheels are advantageously mounted in one plane in a bearing block and mounted in such a way that the axes intersect.

According to a second aspect of the invention, a microscope arrangement is furnished, which first of all provides a retaining device according to the invention as previously described. Therefore, reference is made also to the full content of the above statements regarding the retaining device. In addition, the microscope arrangement provides a microscope which is fastened to the retaining device. Advantageously, a stand may also be provided, to which the retaining device is fastened.

In a particularly advantageous manner, the microscope may involve an operating microscope, which can be used in different medical fields. For example, the operating microscope may involve an ophthalmologic microscope.

The present invention advantageously relates to those configurations in which the drives for moving the apparatus, e.g., a microscope, in particular an operating microscope, are provided in the retaining device. However, embodiments are also possible, wherein the drives are provided in the apparatus itself that is joined with the retaining device.

The invention will now be explained in more detail on the basis of an example of embodiment with reference to the appended drawings. Here:

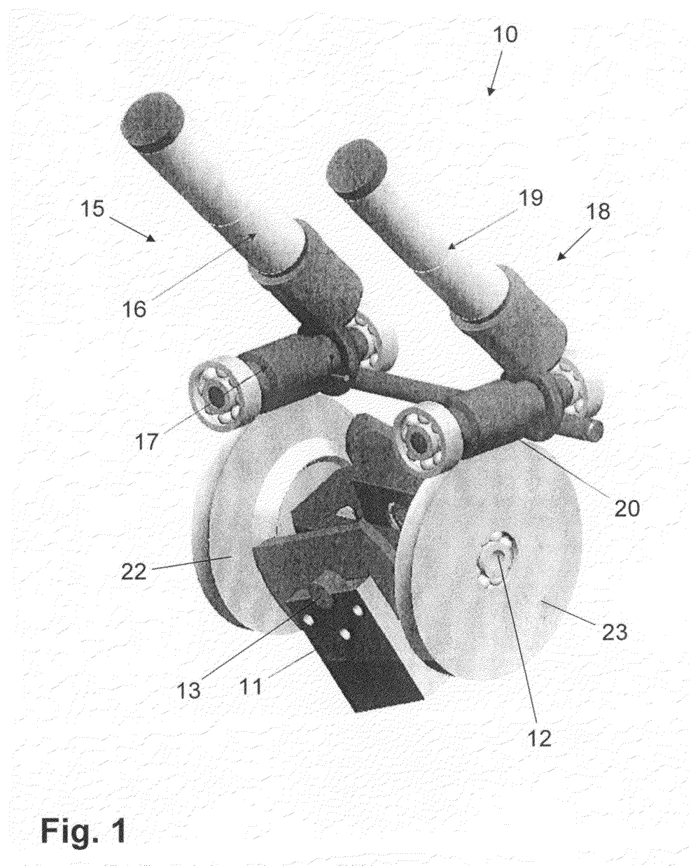

FIG. 1 shows in perspective view a retaining device according to the invention;

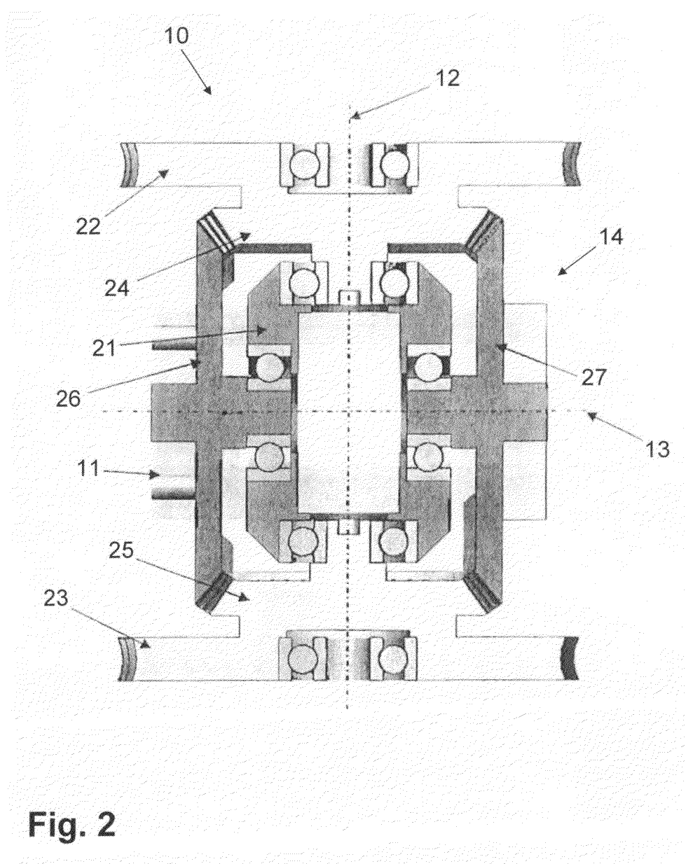

FIG. 2 shows in schematic sectional view a top view onto a first example of embodiment of a gear, which is realized in the retaining device; and

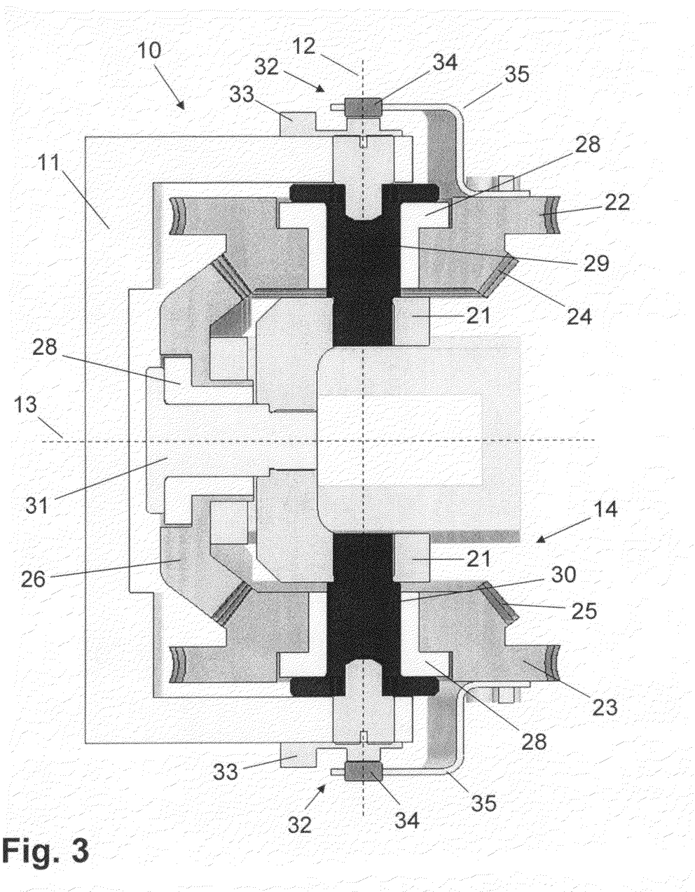

FIG. 3 shows in schematic sectional view a top view onto a second example of embodiment of a gear, which is realized in the retaining device.

FIG. 1 shows a retaining device 10, which provides a fastening means 11 for an apparatus (not shown). In the present example, the fastening means 11 will be designed as a mount for a microscope, for example, an operating microscope.

The fastening means 11 can be tilted via a tilting axis 12 and can be swiveled via a swiveling axis 13. This is essentially executed by means of two worm wheels 22, 23, which are disposed along tilting axis 12. Worm wheels 22, 23 represent components of a gear, which will be explained in more detail in connection with FIG. 2.

Each of worm wheels 22, 23 cooperates with a drive of its own. A first drive 15 provides a motor 16 and a worm drive 17 driven by motor 16. Worm drive 17 cooperates with worm wheel 22. A second drive 18 provides a motor 19 and a worm drive 20 driven by motor 19. Worm drive 20 cooperates with worm wheel 23.

By means of the two drives 15, 18 that can be actuated independently of one another, a tilting of fastening means 11 around tilting axis 12 as well as a swiveling of fastening means 11 around swiveling axis 13 can be carried out. Therefore, two drives 15, 18 carry out the tilting movement, the swiveling movement or a combined movement of the apparatus fastened to fastening means 11. If the two drives 15, 18 rotate synchronously, i.e., in the same direction, then, for example, a tilting of the apparatus, e.g., of the microscope, can be carried out. If the two drives 15, 18 do not rotate synchronously, for example, if one drive rotates counter to the other drive to the same extent, then the apparatus carries out a swiveling movement. If only one of drives 15, 18 rotates, then the apparatus carries out a combined tilting and swiveling movement.

FIG. 2 shows gear 14 of retaining device 10 in greater detail. First, it can be recognized in FIG. 2 that tilting axis 12 and swiveling axis 13 lie in one plane, and that both axes are aligned in such a way that they intersect—in particular orthogonally. Gear 14 can be fastened to a stand (not shown) or similar unit by means of a suitable connection. The microscope (not shown) is fastened to fastening means 11. As has already been explained in connection with FIG. 1, drive 15 propels worm wheel 22, whereas worm wheel 23 is driven by drive 18. Both worm wheels 22, 23 are disposed along tilting axis 12. Further, FIG. 2 shows drive bevel wheels 24, 25, whereby worm wheel 22 with drive bevel wheel 24 and worm wheel 23 with drive bevel wheel 25 are each designed as one part.

Drive bevel wheels 24, 25 are mounted in a bearing block 21. This bearing block 21 is disposed inside gear 14 in this example. It is possible in this way to guide cables and the like through the center of gear 14. Of course, other positions are also possible, for example, all bevel wheels can lie inside bearing block 21 and fastening means 11 can lie in the center. It is only important that tilting axis 12 and swiveling axis 13 intersect, and thus lie in one plane.

In addition, a driven bevel wheel 26 is provided, which is joined in fixed manner with fastening means 11 in the example. In addition, yet another counter bevel wheel 27 is provided, which is mounted on bearing block 21, since driven bevel wheel 26 and counter bevel wheel 27 are asynchronous. Driven bevel wheel 26 and counter bevel wheel 27 are disposed along swiveling axis 13. Counter bevel wheel 27 is not absolutely necessary. It could also be replaced by a mounted shaft, for example.

An essential advantage of retaining device 10 according to the invention with the special drive and the special gear 14 consists of the fact that twice the components will be used. The torque, which acts on tilting axis 12, is divided, for example, onto the two worm wheels 22, 23. Thus, it is not just one worm wheel that bears the torque, but two. If a counter bevel wheel 27 is used, then continually more teeth, for example four, are engaged in order to take up the torque, instead of just one, if only the tilting process is considered. The direction of rotation of the two drives 15, 18 (FIG. 1) alone determines the tilting or swiveling movement.

Another embodiment of a retaining device 10, which has a fastening means 11 for a microscope and which provides a corresponding gear 14, is described in FIG. 3.

The basic structure of retaining device 10 thus corresponds to the retaining device 10 shown in FIG. 2, so that, first of all, reference will be made to the full content of the corresponding statements. In addition, elements that are the same in structure are provided with identical reference numbers.

In comparison to the example shown in FIG. 2, FIG. 3 shows a variant without a counter bevel wheel and with an open bearing block 21. Bearings 28 are designed as combined axial/radial bearings. Tilting axis 12 and swiveling axis 13 in this example of embodiment are designed as structural pieces 29, 30, 31 that can be screwed in, which will be or are tightly screwed with bearing block 21.

The retaining device 10 of FIG. 3 also provides an apparatus 32 for determining position. The position of the microscope disposed on retaining device 10 can be determined by means of this apparatus 32 for determining position. There are various possibilities for accomplishing this purpose. One variant is shown in FIG. 3, in which apparatus 32 for determining position is designed as an encoder device, in particular, as a magnetic encoder device.

The position of worm wheels 22, 23 relative to one another can be detected by means of magnetic encoder device 32. Here, a magnet 34, which is found in each case over a sensor element 33, for example in the form of a sensor IC, is joined in a fixed manner with the respective worm wheel 22, 23 via a magnet holder 35. The microscope position can be determined by interpolating the two encoder values.

### LIST OF REFERENCE NUMBERS

10 Retaining device

11 Fastening means (for a microscope)

12 Tilting axis

13 Swiveling axis

14 Gear

15 Drive

16 Motor

17 Worm drive

18 Drive

19 Motor

20 Worm drive

21 Bearing block

22 Worm wheel

23 Worm wheel

24 Drive bevel wheel

25 Drive bevel wheel

26 Driven bevel wheel

27 Counter bevel wheel

28 Bearing (axial/radial combination bearing)

29 Structural piece

30 Structural piece

31 Structural piece

32 Apparatus for determining position (magnetic encoder device)

33 Sensor element

34 Magnet

35 Magnet holder

1. A retaining device for tilting and swiveling an apparatus, in particular a microscope, which is fastened to retaining device, having a fastening means for fastening the apparatus, a tilting axis for tilting the fastening means and a swiveling axis for swiveling fastening means, hereby characterized in that the tilting axis and the swiveling axis lie in one plane and are aligned relative to one another in such a way that they intersect.

2. The retaining device according to claim 1, further characterized in that in order to conduct a tilting movement of fastening means around tilting axis and in order to conduct a swiveling movement of fastening means around swiveling axis, a single gear is provided.

3. The retaining device according to claim 1, further characterized in that the device has two drives that can be actuated independently of one another, by which means a tilting of fastening means around tilting axis and a swiveling of fastening means around swiveling axis can be carried out.

4. The retaining device according to claim 3, further characterized in that drives are designed so that they can be actuated in a synchronous and/or asynchronous manner.

5. The retaining device according to claim 1, further characterized in that the device has a bearing block, through which the tilting axis and the swiveling axis are extended.

6. The retaining device according to claim 5, further characterized in that the tilting axis and the swiveling axis are designed as structural pieces joined with bearing block.

7. The retaining device according to claim 3, further characterized in that each drive has a worm drive.

8. The retaining device according to claim 2, further characterized in that gear has two worm wheels, which are disposed along the tilting axis.

9. The retaining device according to claim 8, further characterized in that each of worm wheels is joined with a drive bevel wheel.

10. The retaining device according to claim 9, further characterized in that drive bevel wheels are mounted in bearing block.

11. The retaining device according to claim 10, further characterized in that gear has a driven bevel wheel, which is joined in a fixed manner with fastening means.

12. The retaining device according to claim 11, further characterized in that the driven bevel wheel meshes with drive bevel wheels.

13. The retaining device according to claim 5, further characterized in that gear has a counter bevel wheel, which is mounted on bearing block.

14. The retaining device according to claim 1, further characterized in that the device has an apparatus for determining the position of an apparatus fastened to retaining device.

15. The retaining device according to claim 1, further characterized in that the device has an apparatus for fastening to a stand.

16. A microscope arrangement, in particular an operating microscope arrangement, with a retaining device according to one of claims 1 to 15, and with a microscope, which is fastened to retaining device.

17. The microscope arrangement according to claim 16, further characterized in that a stand is provided, to which the retaining device is fastened.
